(19) United States
(12) Patent Application Publication
(10) Pub. No.: US 2021/0100866 A1
(43) Pub. Date: Apr. 8, 2021

(54) METHOD OF TREATMENT USING SYNERGISTIC DIETARY SUPPLEMENTS TO RELIEVE PHYSICAL DISCOMFORT AND PAIN, OR MENTAL DISCOMFORT LINKED TO MENSTRUATION

(71) Applicant: Lun Health Co., Decatur, GA (US)

(72) Inventor: Matthew M. Crane, Decatur, GA (US)

(73) Assignee: Lun Health Co., Decatur, GA (US)

(21) Appl. No.: 17/038,786

(22) Filed: Sep. 30, 2020

Related U.S. Application Data
(60) Provisional application No. 62/909,405, filed on Oct. 2, 2019.

(51) Int. Cl. A61K 36/324; A61K 36/82; A61K 31/353; A61K 33/06; A61K 31/357; A61K 31/05; A61K 31/12; A61K 36/81; A61K 31/593

(52) U.S. Cl. CPC A61K 36/324; A61K 36/82; A61K 31/353; A61K 33/06; A61K 31/593; A61K 31/05; A61K 31/12; A61K 36/81; A61K 31/357

(57) ABSTRACT

Methods of treating dysmenorrhea and bloating, menstruation, headache, backache, and/or intestinal issues associated with menstruation are provided. Menstrual pain treatment systems are further provided.

### CROSS REFERENCE TO RELATED APPLICATIONS

This application claims the benefit of U.S. Provisional Application Ser. No. 62/909,405, filed on Oct. 2, 2019. The disclosure of this prior application is incorporated herein by reference in its entirety for all purposes.

### FIELD OF THE INVENTION

The present invention relates to a method of treating menstrual period pain that links dietary supplementation to immune system changes as a result of the menstrual cycle, so that the supplement is only administered for a period of time overlapping with menstruation. A further method treats those suffering from primary or secondary dysmenorrhea by targeting multiple immune pathways and portions of the inflammatory response through the synergistic use of multiple dietary supplements.

### BACKGROUND

Nutritional supplements on the market are month long, or treated as a one-off for painkillers. Immune system fluctuations and increased inflammation are a direct byproduct of hormonal changes surrounding menstruation. Dietary supplements that reduce menstrual period pain through modified hormone levels have to be taken all month long.

Dietary supplements for dysmenorrhea are given continuously and are not coupled to changes in the immune system. Non-steroidal anti-inflammatory drugs (“nSAIDs”) and other pain relievers are consumed at the time of perceived pain, but are not packaged nor advertised to be consumed prophylactically in advance of menstruation.

Thus, if there were an effective way to alleviate significant pain without a requirement to dose all month which is more convenient for users, and likely to increase adherence rates, this would constitute a useful contribution to the art.

Further, nutritional supplements on the market primarily target hormones. The medical standard of care is to reduce prostaglandin synthesis using nSAIDs that are COX-1/COX-2 inhibitors. Rather than focus just on COX1/COX2, a treatment method that targets multiple aspects of the immune response to generate a synergistic treatment with increased efficacy would constitute a useful contribution to the art.

Accordingly, there exist a need for a combination having multiple targets which results in improved pain relief, even for individuals that respond only slightly to NSAID treatment.

### SUMMARY

According to an example of the present disclosure, a method of treating dysmenorrhea and/or pain associated therewith in a human includes the step of: administering to the human a composition including four or more components including a total combined daily dose of: from about 30 milligrams to about 400 milligrams of Boswellia extract; from about 30 milligrams to about 400 milligrams of green tea catechins; from about 40 milligrams to about 500 milligrams of quercetin; and from about 50 milligrams to about 300 milligrams of magnesium; wherein the composition is administered daily for at least 5 days; wherein the human begins menstruating during a course of administration; and wherein the four or more components act synergistically to treat dysmenorrhea.

According to another example of the present disclosure, a method of treating bloating, headache, backache, anxiety, depression, diarrhea, and/or constipation associated with menstruation in a human includes the step of: administering to the human a composition including four or more components including a total combined daily dose of: from about 30 milligrams to about 400 milligrams of Boswellia extract; from about 30 milligrams to about 400 milligrams of green tea catechins; from about 40 milligrams to about 500 milligrams of quercetin; and from about 50 milligrams to about 300 milligrams of magnesium; wherein the composition is administered daily for at least 5 days; wherein the human begins menstruating during a course of administration; and wherein the four or more components act synergistically to treat bloating, headache, backache, anxiety, depression, diarrhea, and/or constipation.

According to yet another example of the present disclosure, a menstrual pain treatment system includes a single dosage form mixture of four or more components including: from about 30 milligrams to about 400 milligrams of Boswellia extract that has been standardized, and complexed or encapsulated with phospholipids; from about 30 milligrams to about 400 milligrams of green tea catechins that has been standardized, and complexed or encapsulated with phospholipids; from about 40 milligrams to about 500 milligrams of quercetin that has been standardized, and complexed or encapsulated with phospholipids; and from about 50 milligrams to about 300 milligrams of magnesium that has been standardized, and complexed or encapsulated with phospholipids, or from about 50 milligrams to about 300 milligrams magnesium diglycinate or magnesium threonate; and wherein synergistic activity of the mixture is configured to cause, in a menstruating human: a reduction in pelvic pain associated with menstruation; a reduction in bloating associated with menstruation; a reduction in symptoms of anxiety and/or depression associated with menstruation; a reduction in headaches associated with menstruation; a reduction in backaches associated with menstruation; and/or a reduction in constipation or diarrhea associated with menstruation.

According to yet another example of the present disclosure, a method of treating dysmenorrhea in a human includes the step of: administering to the human a composition comprising a mixture of Boswellia extract, green tea catechins, quercetin, magnesium, silybin, resveratrol, curcumin, ashwagandha, and Vitamin D; wherein the composition is administered for at least 5 days; wherein the human begins menstruating during a course of administration; and wherein the four or more components act synergistically to treat dysmenorrhea.

### BRIEF DESCRIPTION OF THE DRAWINGS

The present disclosure may be better understood with reference to the following drawings and description.

FIG. 1 illustrates a plot of supplementation-intervention resultant self-reported pelvic pain during menstruation relative to pain prior to menstruation, as compared to the increase in pelvic pain during menstruation in the absence of supplementation relative to pain prior to menstruation; and

FIG. 2 illustrates a plot of supplementation-intervention resultant decrease in various self-reported pain and/or discomfort types during menstruation.

The drawings described herein are for illustration purposes only and are not intended to limit the scope of the present disclosure in any way.

### DETAILED DESCRIPTION

According to one example of the present disclosure, a method of treating menstrual period pain (including dysmenorrhea), cramps, mood alterations, and/or bloating includes administering a synergistic formulation of dietary supplements to address alterations in the inflammatory response as a result of menstruation. The course of supplements is designed to be taken overlapping with menstruation, and ideally one to two days prior to menstruation. According to another example of the present disclosure, a menstrual pain treatment system includes:

The menstrual cycle is a process characterized by changes in hormone levels. Specifically, the menstrual cycle is a process characterized by fluctuating levels of estrogen and progesterone. As both estrogen and progesterone fall in the days following ovulation, this results in the transition from the luteal to follicular phase of the menstrual cycle, and eventually menstruation. Discomfort or pain during menstruation is one of the most common concerns presented to doctors. Discomfort as a result of the menstrual cycle, either during menstruation or ovulation, is largely described as result of hormonal imbalance, and pharmacological and medical interventions focus on hormones. The description of discomfort as a result of the menstrual cycle as resulting from hormonal imbalance, however, is not completely accurate as there are significant changes to the immune system during progression through the menstrual cycle, and these immune system changes have been strongly linked to period pain.

The standard pharmacological interventions primarily rely on forms of hormonal contraception which provides subjects with consistent levels of sex hormones. Hormonal contraception has been found to mitigate discomfort and pain related to menstruation. Because high doses of progestin can have negative side-effects including weight gain, acne, and altered blood clotting in individuals with higher risk of strokes, hormonal contraception includes both estrogen and progestin. In cases of secondary dysmenorrhea (when a physical cause of menstrual pain has been identified), there are also potential surgical solutions that can be employed.

Non-hormonal pharmacological interventions to treat period pain are largely centered around non-steroidal anti-inflammatory drugs (“nSAIDs”) that inhibit the cyclooxygenase pathway and reduce prostaglandin synthesis. Examples of nSAIDs may include ibuprofen and naproxen sodium. Side-effects of nSAIDs may include irritation of the stomach lining, which can lead to ulcers or stomach bleeding. Additionally, about 20% of women experience minimal or no reduction in period pain as a result of NSAID treatment.

Significant work has also gone into the development of gonadotropin-releasing hormone (“GnRH”) receptor antagonists. GnRH receptor antagonists can have significant benefits in the treatment of pain associated with secondary dysmenorrhea, such as endometriosis, but are associated with significant side effects. The most common side effects are peri-menopausal symptoms including hot flashes, night sweats and nausea. More serious side effects are a reduction in bone-mineral density (“BMD”) and alterations to the lipid profile. Given the vulnerability of women to osteoporosis and the severe consequences of fractures in old age, reductions in BMD are particularly concerning, and treatment with GnRH receptor antagonists is not recommended to continue for years.

In the nutraceutical or dietary supplement space, research and product development has primarily, if not exclusively, focused on similar approaches that affect hormones. Several of the most popular and commonly used supplements for menstrual pain are chasteberry (Vitex agnus-castus), dong quai (Angelica sinensis), black cohosh (Cimicifuga racemosa), and maca (Lepidium meyenii). All of these methods of treatment are designed to influence levels of estrogen and progesterone, and as a result, to modulate period pain or affect polycystic ovarian syndrome (“PCOS”).

The supplements currently used for menstrual pain have potential drawbacks and few studies have suggested that the currently used supplements are necessarily useful for treating dysmenorrhea or other common menstrual related physiological changes resulting in discomfort. Dong quai and black cohosh both have estrogenic activities and patients with hormone sensitive cancers (especially estrogen receptor positive cancers) are recommended to avoid dong quai and black cohosh. Maca has also been shown to increase lutenizing hormones (“LH”) in mouse models, and appears to affect testosterone levels in humans. Chasteberry appears to restore LH levels, increase progesterone levels, and has been clinically shown to reduce menstrual pain when taken continuously all month long. Because chasteberry does have phytoestrogen properties, individuals with hormone sensitive cancers should avoid chasteberry.

In spite of the significant focus on hormonal modifications and treatments to treat dysmenorrhea, there is significant evidence that immune system alterations are significant. Higher levels of inflammatory markers have been correlated with higher levels of dysmenorrhea. Further, research into a secondary dysmenorrhea disease, endometriosis, has identified numerous alterations to the immune system that influence disease progression and pain. For example, patients with endometriosis are likely to have altered natural killer cell behavior, and macrophages with increased levels of IGF-1 secretion. Additionally, there is a positive feedback loop between the inflammatory state and estrogen production, which can then result in increasing levels of inflammation. Although the focus has been on hormonal disruptors (for example, using Chasteberry to increase progesterone levels) to break this cycle, a reduced inflammatory response could perform a similar role.

Furthermore, there is considerable research into a connection between intestinal issues (specifically irritable bowel syndrome (“IBS”)) and dysmenorrhea. The link between IBS and dysmenorrhea is not fully understood, but the link is believed to result from a feedback between the inflammatory states of both IBS and dysmenorrhea. In subjects with IBS, there is an increase in inflammatory cytokines and prostaglandins in and surrounding the intestines. The increase in inflammatory cytokines and prostaglandins affects the pelvic cavity, and during menstruation there is also an increase in inflammatory markers (prostaglandins) as a result of the breakdown in the uterine lining. This feedback between the increase in inflammatory markers and breakdown in the uterine lining is believed to increase both period pain and IBS symptoms. The link between IBS and dysmenorrhea has been neglected as a treatment option heretofore.

Furthermore, numerous studies have shown that there is a significant increase in oxidative stress levels in women with increased period pain, and that alleviating oxidative stress can help reduce menstrual pain. The link between oxidative stress and menstrual pain has been neglected as a treatment option heretofore.

Various aspects will be described in detail hereinafter. Such aspects may, however, be exemplified in many different forms and the examples should not be construed as limited to those set forth herein; rather, these examples are provided so that this disclosure will be thorough and complete, and will fully convey its scope to those skilled in the art. For convenience, certain terms employed in the specification are collected here. Unless defined otherwise, all technical and scientific terms used in this disclosure have the same meanings as commonly understood by one of ordinary skill in the art to which this disclosure belongs.

Where a range of values is provided, it is intended that each intervening integer value between the upper and lower limit of that range and any other stated or intervening value in that stated range is encompassed within the disclosure. For example, if a range of 10 mg to 20 mg is stated, it is intended that 11 mg, 12 mg, 13 mg, 14 mg, 15 mg, 16 mg, 17 mg, 18 mg, and 19 mg are also explicitly disclosed, as well as the range of values greater than or equal to 10 mg and the range of values less than or equal to 20 mg.

As used herein the term “about” means plus or minus 10% of the value being discussed. Therefore, about 100 mg means a range from 90 mg to 110 mg.

The compositions of a treatment system are preferably in unit dosage forms, or unit nutritional or dietary supplement forms, namely servings. In such form, the preparation is subdivided into unit doses or servings containing appropriate quantities of the active component(s). The unit dosage form may be a packaged preparation, the package containing discrete quantities of preparation, such as packaged cooked pieces, tablets, capsules, and powders in vials or ampoules. Also, the unit dosage form may be a capsule, tablet, cachet, or lozenge itself, or it can be the appropriate number of any of these in packaged form.

The term “dysmenorrhea,” as used herein, unless stated otherwise, alone or in combination with other terms, refers to a uterine contractile event, in which the uterus contracts and relaxes with sufficient force to cause reduced blood supply to the uterus, reducing oxygen, and resulting in pain. Dysmenorrhea is classified as primary (spontaneous onset) or secondary (due to some inciting event). In addition to painful uterine contractions with menses, women with dysmenorrhea may experience nausea, vomiting, diarrhea, headaches, weakness, and/or fainting. Symptoms may vary in severity from cycle to cycle, but generally continue throughout the reproductive years.

The terms “synergy,” “synergic,” “synergistic,” and “synergistically,” as used herein, unless stated otherwise, alone or in combination with other terms, refer to an interaction between two or more active ingredients that causes the total effect of the two or more active ingredients to be greater than the sum of the individual effects of each individual active ingredient.

The terms “treatment,” “treat,” and “treating,” as used herein, unless stated otherwise, alone or in combination with other terms, refer to any treatment of a disease or disorder, in a mammal, including: arresting or suppressing the development of clinical symptoms; and/or causing the regression of clinical symptoms.

Delivery or Treatment System

Suitable dosage forms include tablets, capsules, solutions, suspensions, powders, gums, and confectionaries. Sublingual delivery systems include, but are not limited to, dissolvable tabs under and on the tongue, liquid drops, and beverages. Edible films, hydrophilic polymers, oral dissolvable films, or oral dissolvable strips may be used. Other useful delivery systems include oral or nasal sprays or inhalers, and the like.

For oral administration, the composition may be combined with one or more solid inactive ingredients for the preparation of tablets, capsules, pills, powders, granules, or other suitable dosage forms.

For example, the active agent may be combined with at least one excipient such as fillers, binders, emulsifiers, humectants, disintegrating agents, solution retarders, absorption accelerators, wetting agents, absorbents, or lubricating agents. Other useful excipients include magnesium stearate, calcium stearate, calcium sulfate, mannitol, xylitol, sweeteners, starch, carboxymethylcellulose, microcrystalline cellulose, silica, gelatin, silicon dioxide, and the like. Other useful excipients include: carrier materials such as starch, gelatin, acacia, microcrystalline cellulose, kaolin, dicalcium phosphate, calcium carbonate, sodium chloride, alginic acid, and the like; disintegrators including microcrystalline cellulose, alginic acid, and the like; binders including acacia, methylcellulose, sodium carboxymethylcellulose, polyvinylpyrrolidone, hydroxypropyl methylcellulose, ethyl cellulose, and the like; and lubricants or flow agents such as magnesium stearate, calcium stearate, stearic acid, silicone fluid, talc, waxes, oils, colloidal silica, and the like. The usefulness of such excipients is well known in the art.

Nutraceutical compositions are contemplated and may be administered in combination with a nutraceutically acceptable carrier. The active ingredients in such formulations may comprise from 1% by weight to 99% by weight, or alternatively, 0.1% by weight to 99.9% by weight. “Nutraceutically acceptable carrier” means any carrier, diluent or excipient that is compatible with the other ingredients of the formulation and not deleterious to the user.

Liquid nutritional compositions for oral administration may be prepared in water, juices, or other aqueous vehicles. In addition to the above enumerated ingredients or compounds, liquid nutritional compositions may include suspending agents such as, for example, methylcellulose, alginates, tragacanth, pectin, kelgin, carrageenan, acacia, polyvinylpyrrolidone, polyvinyl alcohol, and the like. The liquid nutritional compositions may be in the form of a solution, emulsion, syrup, gel, or elixir including or containing, together with the above enumerated ingredients or compounds, wetting agents, sweeteners, and coloring and flavoring agents. Various liquid and powder nutritional compositions may be prepared by conventional methods.

Routes of Administration

The compounds may be administered by any route, including but not limited to oral, sublingual, buccal, ocular, pulmonary, rectal, and parenteral administration, or as an oral or nasal spray (for example, inhalation of nebulized vapors, droplets, or solid particles). Parenteral administration includes, for example, intravenous, intramuscular, intraarterial, intraperitoneal, intranasal, intravaginal, intravesical (for example, to the bladder), intradermal, transdermal, topical, or subcutaneous administration. Also contemplated within the scope of the invention is the instillation of pterostilbene in the body of the patient in a controlled formulation, with systemic or local release of the drug to occur at a later time. For example, the drug may be localized in a depot for controlled release to the circulation, or for release to a local site of tumor growth.

The treatment may be carried out for as long a period as necessary, either in a single, uninterrupted session, or in discrete sessions. The treating physician will know how to increase, decrease, or interrupt treatment based on patient response. According to one example, treatment is carried out for from about four to about twelve weeks, or, in other words, for two or more consecutive menstrual cycles. The treatment schedule may be repeated as required.

The course of supplements is simple, and straightforward. By focusing on a single 5-10 day course of supplements around menstruation, the course of supplements increases adherence and ease for the consumer. The supplement design focuses on immune system variations as a result of menstruation and focuses on multiple aspects of the immune system and physiology rather than a single aspect which improves outcomes.

An exemplary formulation has been shown to statistically significantly lower self-reported pelvic pain, bloating, headaches, negative emotions. The exemplary formulation has also been shown to statistically significantly improve ability to engage in normal social activities (see the attached intervention study design and results). Furthermore, one subject who reported that nSAIDs had never reduced her period pain found that the exemplary formulations disclosed herein did reduce her period pain, which was likely due to the synergistic nature of the ingredients and the focus on multiple aspects of the immune and inflammatory response.

The course of supplements or menstrual pain treatment system is simple, and straightforward. Rather than being consumed continuously as dietary supplements that focus on hormonal changes require, the course of supplements or treatment system is a simple 5-10 day course which is taken during and surrounding menstruation. Menstruation already requires specific processes and modification to the daily routine, so a course of supplements or treatment system specifically for the time during and surrounding menstruation requires minimal modification to behaviors, and will increase compliance.

The supplement design or treatment system focuses on immune system variations as a result of menstruation. This design makes the supplement more generally applicable to people experiencing increased pain or discomfort during menstruation. Rather than attempting to address hormonal variations, which may need to be customized, the supplement focuses on specific aspects of the immune and inflammatory response during the menstrual cycle.

The formulation focuses on multiple aspects of the immune system and physiology. Rather than just addressing prostaglandin synthesis through inhibition of the cyclooxygenase pathway, as is the standard of care through the use of nSAIDs, the formulation uses compounds shown to affect multiple inflammatory aspects. The effects on inflammatory aspects may include the inhibition of cyclooxygenase, inhibition of lipoxygenase, stabilization of mast cells, and reduction in inducible nitric oxide synthase. Furthermore, by including a magnesium species and cholecalciferol (Vitamin D3), the formulation ameliorates nutritional deficiencies that have been linked to increased pain and discomfort during menstruation. Accordingly, the formulation or treatment system may be configured to inhibit cyclooxygenase, inhibit lipoxygenase, stabilize mast cells, reduce inducible nitric oxide synthase, and ameliorate nutritional deficiencies.

Administration of single isolated compounds to a test subject was not found to significantly ameliorate pain and discomfort during menstruation. During separate menstrual cycles, a single test subject was provided with separate treatments of: 1) curcumin; 2) curcumin and magnesium; 3) curcumin, resveratrol, and magnesium; and 4) curcumin, resveratrol, silybin, magnesium, ashwagandha, vitamin D3, quercetin, boswellia, and green tea catechins. All treatments were supplied at dosages used in the initial intervention study. Significantly, the combination of all ingredients was reported to significantly affect pain, bloating and mood during menstruation. In examples of methods and systems of the present disclosure, treatment with curcumin, resveratrol, a magnesium species, and one or more of at least silybin, ashwagandha, vitamin D3, quercetin, boswellia, and/or green tea catechins will provide synergistic effects on reduction of self-reported period or pelvic pain, bloating, headaches, intestinal discomfort, negative emotions, and/or inability to engage normally in social activities. In certain examples, the synergism exhibited by the combination of ingredients may be such that doses of the individual ingredients would be sub-therapeutic if administered separately. The synergy exhibited by four ingredients may be further augmented by addition of a fifth, sixth, seventh, eighth, or ninth ingredient.

In an intervention study of 10 women with self-reported pelvic pain, there was a significant reduction in pain associated with multiple physiological symptoms (pelvic pain, bloating, headaches) as well as improved mood.

In a second intervention study of 14 women with self-reported pelvic pain, there was a significant reduction in pain associated with multiple physiological symptoms (for example, pelvic pain, bloating, headaches) as well as improved mood and an ability to perform routine daily activities (for example, meet with friends, exercise) during menstruation.

One example of a method to treat cramps, bloating, and/or dysmenorrhea includes administering to a user an anti-inflammatory dietary supplement formulation for a duration of 5-15 days that overlaps with menstruation. Ideally the duration begins prior to or coincident with the beginning of menstruation.

Another example of a method to treat cramps, bloating, and/or dysmenorrhea includes administering formulations with 4 or more of the following core ingredients, for the purpose of alleviating or diminishing pain associated with hormonal changes during the menstrual cycle, and configured to alleviate or diminish pain associated with hormonal changes during the menstrual cycle. In certain examples, the synergism exhibited by the combination of four ingredients may be such that doses of the individual ingredients would be sub-therapeutic if administered separately. The synergy exhibited by the combination of four ingredients may be further augmented by addition of a fifth, sixth, seventh, eighth, or ninth ingredient.

Boswellia serrata—Boswellia has multiple active components, each of which is a triterpenoid acid. These are β-boswellic acid (“β-BA”), acetyl-β-boswellic acid (“β-ABA”), 11-keto-β-boswellic acid (“β-KBA”) and acetyl-11-keto-β-boswellic acid (“β-AKBA”). Both β-KBA and β-AKBA are selective 5-lipoxygenase (“5-lox”) inhibitors. Inhibition of 5-lox reduces the inflammatory response by reducing leukotriene levels. In women with primary dysmenorrhea who do not respond to prostaglandin antagonists (nSAIDs), significant levels of leukotrienes have been found in the endometria. The selective inhibition of 5-lox is relatively unique among botanical extracts. β-BA has also been found to inhibit cathepsin g and microsomal prostglandin E synthase 1. Studies of the triterpenoid acids have found β-BA to significantly reduce IBS and intestinal distress, which is a comorbidity with dysmenorrhea. Supplementation with Boswellia may be configured to reduce IBS and intestinal distress.

Curcumin—Curcumin is an extract from Curcuma longa that has been extensively studied for curcumin's anti-oxidant and anti-inflammatory properties. Not only has curcumin has been shown to significantly lower inflammation in numerous mechanistic studies, but a large number of clinical studies have shown benefits in patients with increased inflammation (for example osteoarthritis). Curcumin also increases antioxidant activity by reducing glutathione and increase superoxide dismutase (SOD). Curcumin also inhibits matrix-metalloproteinases (“MMPs”) which help breakdown tissues as part of healing as well as angiogenesis. The inhibition of MMPs is an important aspect of lesion growth for some secondary dysmenorrhea (endometriosis) as increases in levels of MMPs in the pelvis are thought to assist in endometrial lesion invasion. Curcumin has been studied in multiple pre-clinical animal models, and curcumin appears to significantly reduce endometrial lesion size and growth.

Resveratrol (and/or Pterostilbene)—Resveratrol and pterostilbene, which is a dimethylated form of resveratrol, are stilbenoids found primarily in the skin of fruits such as grapes or blueberries. Resveratrol has been shown to have multiple active roles, including inhibition of NF-kB, and multiple antioxidant roles (including increased glutathione and increased super oxide dismutase). Resveratrol has also been shown to reduce levels of prostaglandin E2 as a result of cyclooxygenase inhibition. Resveratrol also inhibits MMP-2, which has been shown to reduce the growth of endometrial lesions. Pterostilbene has been less studied than resveratrol, but the mechanisms of action are believed to be the same; because pterostilbene is dimethylated, pterostilbene has a significantly increased half-life.

Ashwagandha—Ashwagandha (Withania somnifera) has primarily been studied as part of methods to reduce stress. There are a number of high-quality clinical trials that have shown Ashwagandha reduces anxiety and stress, and improves subjective well-being. In the medium term, the reduction in stress levels has been linked to lower cortisol levels and a reduction in C-reactive protein, a blood measure of inflammation.

Green tea catechins—Catechins are a group of flavan-3-ols that are primarily found in teas produced from the leaves of Camellia sinensis. The most widely studied catechin is Epigallocatechin-3-gallate (“EGCG”), but there multiple catechins, and recent studies have suggested that catechins may act synergistically. Tea catechins have been shown to be anti-inflammatory by reducing cyclooxygenase activity by inhibition of Nf-kB. Additionally, catechins have been investigated from the perspective of secondary dysmenorrhea (specifically endometriosis) due to the ability of catechins to inhibit angiogenesis. Angiogenesis, or the growth of new blood vessels is important for the progression of endometriosis, and green tea catechins have shown to inhibit endometrial lesion growth in several pre-clinical mouse studies. Tea catechins inhibit vascular endothelial growth factor (VEGF) which is critical to angiogenesis.

Quercetin—Quercetin is a flavonol found in many fruits and vegetables. Quercetin is one of the most frequently studied flavonoids, and has many biological roles. Quercetin acts as an antioxidant through direct scavenging of free-radicals, promotion of glutathione, and increased levels of SOD. Quercetin also acts in as an anti-inflammatory by inhibiting cyclooxygenase and reducing prostaglandin levels. Importantly, quercetin also blocks the release of histamine by stabilizing mast cells, and has been studied as part of a method to reduce asthma by reducing histamine release.

Silybin—Extracts from milk thistle (Silybum marianum) have historically been used for treating the liver. Milk thistle extract includes multiple active ingredients, and silymarin is a mixture of multiple polyphenolic molecules. The most active of these polyphenolic molecules is thought to be silybin. Silybin has antioxidant activities, stabilizes mast cells, and inhibits both cyclooxygenase and lipoxygenase. Furthermore, silybin has been shown to limit angiogenesis in cancer models, which could help reduce the spread of endometrial lesions in secondary dysmenorrhea. In a mouse model of endometriosis, silybin resulted in reduced endometrial lesion sizes relative to control.

Magnesium—Magnesium is an essential dietary mineral, but one that is becoming increasingly rare in modern diets. In addition to changing dietary habits, which has reduced consumption of magnesium, modern farming practices and climate change have reduced magnesium levels in most vegetables. As a result, ˜70% of people in the United States consume less than the recommended dietary allowance of magnesium. Supplementation with a magnesium species has been clinically shown to reduce dysmenorrhea, possibly as result of alleviating dietary deficiencies. Higher magnesium levels may also reduce muscle cramps. Supplementation with magnesium may be configured to reduce dysmenorrhea and/or muscle cramps.

Vitamin D—Vitamin D is a group of fat-soluble secosteroids that help with absorption of several critical minerals including calcium and magnesium. The two most important forms for humans are ergocalciferol (Vitamin D2) and cholecalciferol (Vitamin D3). Both can be obtained through dietary sources, but at very low levels. Cholecalciferol is made in the skin in response to exposure to sunlight. Low vitamin D levels have been linked to increased prevalence of dysmenorrhea, and supplementation to alleviate the deficiency has been shown to reduce period pain. Vitamin D supplementation may be configured to reduce period pain. It is not known whether additional supplementation in individuals with sufficient Vitamin D levels is beneficial at reducing dysmenorrhea. One proposed mechanism of action is that sufficient vitamin D levels helps promote mast cell stabilization.

In an example, the formulation may include the following components:

A preferred daily dosage range of from about 20 mg to about 300 mg of silybin (Silymarin), or a most preferred dosage range of from about 20 mg to about 80 mg (about 60 mg tested in Study 1 and about 44 mg in Study 2), including, but not limited to, from about 20 mg to about 290 mg, from about 30 to about 290 mg, from about 40 to about 290 mg, from about 50 mg to about 290 mg, from about 60 to about 290 mg, from about 20 mg to about 100 mg, from about 20 mg to about 110 mg, from about 20 mg to about 120 mg, from about 20 mg to about 130 mg, from about 20 mg to about 140 mg, from about 20 mg to about 150 mg, from about 20 mg to about 160 mg, from about 20 mg to about 170 mg, from about 20 mg to about 180 mg, from about 20 mg to about 190 mg, from about 20 mg to about 200 mg, from about 20 mg to about 210 mg, from about 20 mg to about 220 mg, from about 20 mg to about 230 mg, from about 20 mg to about 240 mg, from about 20 mg to about 250 mg, from about 20 mg to about 260 mg, from about 20 mg to about 270 mg, from about 20 mg to about 280 mg, or from about 20 mg to about 290 mg of silybin;

A preferred daily dosage range of from about 30 mg to about 400 mg of Boswellia extract, or a most preferred dosage range of from about 40 mg to about 140 mg (about 83 mg tested in Study 1 and 63 mg in Study 2), including, but not limited to, from about 50 mg to about 390 mg, from about 60 mg to about 390 mg, from about 70 mg to about 390 mg, from about 80 mg to about 390 mg, from about 90 mg to about 390 mg, from about 40 mg to about 150 mg, from about 40 mg to about 160 mg, from about 40 mg to about 170 mg, from about 40 mg to about 180 mg, from about 40 mg to about 190 mg, from about 40 mg to about 200 mg, from about 40 mg to about 210 mg, from about 40 mg to about 220 mg, from about 40 mg to about 230 mg, from about 40 mg to about 240 mg, from about 40 mg to about 250 mg, from about 40 mg to about 260 mg, from about 40 mg to about 270 mg, from about 40 mg to about 280 mg, from about 40 mg to about 290 mg, from about 40 mg to about 300 mg, from about 40 mg to about 310 mg, from about 40 mg to about 320 mg, from about 40 mg to about 330 mg, from about 40 mg to about 340 mg, from about 40 mg to about 350 mg, from about 40 mg to about 360 mg, from about 40 mg to about 370 mg, from about 40 mg to about 380 mg, or from about 40 mg to about 390 mg Boswellia extract;

A preferred daily dosage range of from about 30 mg to about 400 mg of resveratrol, or a most preferred dosage range of from about 40 mg to about 300 mg (about 75 mg tested in Study 1 and about 247 mg in Study 2), including, but not limited to, from about 50 mg to about 390 mg, from about 60 mg to about 390 mg, from about 70 mg to about 390 mg, from about 80 mg to about 390 mg, from about 90 mg to about 390 mg, from about 100 mg to about 390 mg, from about 110 mg to about 390 mg, from about 120 mg to about 390 mg, from about 130 mg to about 390 mg, from about 140 mg to about 390 mg, from about 150 mg to about 390 mg, from about 160 mg to about 390 mg, from about 170 mg to about 390 mg, from about 180 mg to about 390 mg, from about 190 mg to about 390 mg, from about 200 mg to about 390 mg, from about 210 mg to about 390 mg, from about 220 mg to about 390 mg, from about 230 mg to about 390 mg, from about 240 mg to about 390 mg, from about 250 mg to about 390 mg, from about 40 mg to about 310 mg, from about 40 mg to about 320 mg, from about 40 mg to about 330 mg, from about 40 mg to about 340 mg, from about 40 mg to about 350 mg, from about 40 mg to about 360 mg, from about 40 mg to about 370 mg, from about 40 mg to about 380 mg, or from about 40 mg to about 390 mg resveratrol;

A preferred daily dosage range of from about 30 mg to about 400 mg of green tea catechins, or a most preferred dosage range of from about 40 mg to about 200 mg (about 83 mg tested in Study 1 and about 120 mg in Study 2), including, but not limited to, from about 50 mg to about 390 mg, from about 60 mg to about 390 mg, from about 70 mg to about 390 mg, from about 80 mg to about 390 mg, from about 90 mg to about 390 mg, from about 100 mg to about 390 mg, from about 110 mg to about 390 mg, from about 120 mg to about 390 mg, from about 40 mg to about 210 mg, from about 40 mg to about 220 mg, from about 40 mg to about 230 mg, from about 40 mg to about 240 mg, from about 40 mg to about 250 mg, from about 40 mg to about 260 mg, from about 40 mg to about 270 mg, from about 40 mg to about 280 mg, from about 40 mg to about 290 mg, from about 40 mg to about 300 mg, from about 40 mg to about 310 mg, from about 40 mg to about 320 mg, from about 40 mg to about 330 mg, from about 40 mg to about 340 mg, from about 40 mg to about 350 mg, from about 40 mg to about 360 mg, from about 40 mg to about 370 mg, from about 40 mg to about 380 mg, or from about 40 mg to about 390 mg green tea catechins;

A preferred daily dosage range of from about 40 mg to about 500 mg of quercetin, or a most preferred dosage range of from about 50 mg to about 200 mg (about 148 mg tested in Study 1 and about 96 mg in Study 2), including, but not limited to, from about 60 mg to about 490 mg, from about 70 mg to about 490 mg, from about 80 mg to about 490 mg, from about 90 mg to about 490 mg, from about 90 mg to about 490 mg, from about 100 mg to about 490 mg, from about 110 mg to about 490 mg, from about 120 mg to about 490 mg, from about 130 mg to about 490 mg, from about 140 mg to about 490 mg, from about 150 mg to about 490 mg, from about 50 mg to about 210 mg, from about 50 mg to about 220 mg, from about 50 mg to about 230 mg, from about 50 mg to about 240 mg, from about 50 mg to about 250 mg, from about 50 mg to about 260 mg, from about 50 mg to about 270 mg, from about 50 mg to about 280 mg, from about 50 mg to about 290 mg, from about 50 mg to about 300 mg, from about 50 mg to about 310 mg, from about 50 mg to about 320 mg, from about 50 mg to about 330 mg, from about 50 mg to about 340 mg, from about 50 mg to about 350 mg, from about 50 mg to about 360 mg, from about 50 mg to about 370 mg, from about 50 mg to about 380 mg, from about 50 mg to about 390 mg, from about 50 mg to about 400 mg, from about 50 mg to about 410 mg, from about 50 mg to about 420 mg, from about 50 mg to about 430 mg, from about 50 mg to about 440 mg, from about 50 mg to about 450 mg, from about 50 mg to about 460 mg, from about 50 mg to about 470 mg, from about 50 mg to about 480 mg, or from about 50 mg to about 490 mg quercetin;

A preferred daily dosage range of from about 50 mg to about 600 mg of curcumin, or a most preferred dosage range of from about 70 mg to about 200 mg (about 130 mg tested in Study 1 and about 117 mg in Study 2), including, but not limited to, from about 60 mg to about 590 mg, from about 80 mg to about 590 mg, from about 90 mg to about 590 mg, from about 100 mg to about 590 mg, from about 110 mg to about 590 mg, from about 120 mg to about 590 mg, from about 130 mg to about 590 mg, from about 70 mg to about 210 mg, from about 70 mg to about 220 mg, from about 70 mg to about 230 mg, from about 70 mg to about 240 mg, from about 70 mg to about 250 mg, from about 70 mg to about 260 mg, from about 70 mg to about 270 mg, from about 70 mg to about 280 mg, from about 70 mg to about 290 mg, from about 70 mg to about 300 mg, from about 70 mg to about 310 mg, from about 70 mg to about 320 mg, from about 70 mg to about 330 mg, from about 70 mg to about 340 mg, from about 70 mg to about 350 mg, from about 70 mg to about 360 mg, from about 70 mg to about 370 mg, from about 70 mg to about 380 mg, from about 70 mg to about 390 mg, from about 70 mg to about 400 mg, from about 70 mg to about 410 mg, from about 70 mg to about 420 mg, from about 70 mg to about 430 mg, from about 70 mg to about 440 mg, from about 70 mg to about 450 mg, from about 70 mg to about 460 mg, from about 70 mg to about 470 mg, from about 70 mg to about 480 mg, from about 70 mg to about 490 mg, from about 70 mg to about 500 mg, from about 70 mg to about 510 mg, from about 70 mg to about 520 mg, from about 70 mg to about 530 mg, from about 70 mg to about 540 mg, from about 70 mg to about 550 mg, from about 70 mg to about 560 mg, from about 70 mg to about 570 mg, from about 70 mg to about 580 mg, or from about 70 mg to about 590 mg curcumin;

A preferred daily dosage range of from about 70 mg to about 600 mg of ashwagandha, or a most preferred dosage range of from about 80 mg to about 400 mg (about 250 mg tested in Study 1 and about 132 mg in Study 2), including, but not limited to, from about 90 mg to about 590 mg, from about 100 mg to about 590 mg, from about 110 mg to about 590 mg, from about 120 mg to about 590 mg, from about 130 mg to about 590 mg, from about 130 mg to about 590 mg, from about 140 mg to about 590 mg, from about 150 mg to about 590 mg, from about 160 mg to about 590 mg, from about 170 mg to about 590 mg, from about 180 mg to about 590 mg, from about 190 mg to about 590 mg, from about 200 mg to about 590 mg, from about 210 mg to about 590 mg, from about 220 mg to about 590 mg, from about 230 mg to about 590 mg, from about 240 mg to about 590 mg, from about 250 mg to about 590 mg, from about 80 mg to about 410 mg, from about 80 mg to about 420 mg, from about 80 mg to about 430 mg, from about 80 mg to about 440 mg, from about 80 mg to about 450 mg, from about 80 mg to about 460 mg, from about 80 mg to about 470 mg, from about 80 mg to about 480 mg, from about 80 mg to about 490 mg, from about 80 mg to about 500 mg, from about 80 mg to about 510 mg, from about 80 mg to about 520 mg, from about 80 mg to about 530 mg, from about 80 mg to about 540 mg, from about 80 mg to about 550 mg, from about 80 mg to about 560 mg, from about 80 mg to about 570 mg, from about 80 mg to about 580 mg, or from about 80 mg to about 590 mg ashwagandha;

A preferred daily dosage range of from about 1,000 IU to about 10,000 IU Vitamin D, or a most preferred dosage range of from about 2,000 IU to about 7,000 IU (about 4,000 IU tested of Vitamin D3 in Study 1 and about 4,800 IU in Study 2), including, but not limited to, from about 1,100 IU to about 9,900 IU, from about 1,200 IU to about 9,900 IU, from about 1,300 IU to about 9,900 IU, from about 1,400 IU to about 9,900 IU, from about 1,500 IU to about 9,900 IU, from about 1,600 IU to about 9,900 IU, from about 1,700 IU to about 9,900 IU, from about 1,800 IU to about 9,900 IU, from about 1,900 IU to about 9,900 IU, from about 2,100 IU to about 9,900 IU, from about 2,200 IU to about 9,900 IU, from about 2,300 IU to about 9,900 IU, from about 2,400 IU to about 9,900 IU, from about 2,500 IU to about 9,900 IU, from about 2,600 IU to about 9,900 IU, from about 2,700 IU to about 9,900 IU, from about 2,800 IU to about 9,900 IU, from about 2,900 IU to about 9,900 IU, from about 3,000 IU to about 9,900 IU, from about 3,100 IU to about 9,900 IU, from about 3,200 IU to about 9,900 IU, from about 3,300 IU to about 9,900 IU, from about 3,400 IU to about 9,900 IU, from about 3,500 IU to about 9,900 IU, from about 3,600 IU to about 9,900 IU, from about 3,700 IU to about 9,900 IU, from about 3,800 IU to about 9,900 IU, from about 3,900 IU to about 9,900 IU, from about 4,000 IU to about 9,900 IU, from about 4,100 IU to about 9,900 IU, from about 4,200 IU to about 9,900 IU, from about 4,300 IU to about 9,900 IU, from about 4,400 IU to about 9,900 IU, from about 4,500 IU to about 9,900 IU, from about 4,600 IU to about 9,900 IU, from about 4,700 IU to about 9,900 IU, from about 4,800 IU to about 9,900 IU, from about from about 2,000 IU to about 7,100 IU, from about 2,000 IU to about 7,200 IU, from about 2,000 IU to about 7,300 IU, from about 2,000 IU to about 7,400 IU, from about 2,000 IU to about 7,500 IU, from about 2,000 IU to about 7,600 IU, from about 2,000 IU to about 7,700 IU, from about 2,000 IU to about 7,800 IU, from about 2,000 IU to about 7,900 IU, from about 2,000 IU to about 8,000 IU, from about 2,000 IU to about 8,100 IU, from about 2,000 IU to about 8,200 IU, from about 2,000 IU to about 8,300 IU, from about 2,000 IU to about 8,400 IU, from about 2,000 IU to about 8,500 IU, from about 2,000 IU to about 8,600 IU, from about 2,000 IU to about 8,700 IU, from about 2,000 IU to about 8,800 IU, from about 2,000 IU to about 8,900 IU, from about 2,000 IU to about 9,000 IU, from about 2,000 IU to about 9,100 IU, from about 2,000 IU to about 9,200 IU, from about 2,000 IU to about 9,300 IU, from about 2,000 IU to about 9,400 IU, from about 2,000 IU to about 9,500 IU, from about 2,000 IU to about 9,600 IU, from about 2,000 IU to about 9,700 IU, from about 2,000 IU to about 9,800 IU, or from about 2,000 IU to about 9,900 IU Vitamin D; and

A preferred daily dosage range of from about 50 mg to about 300 mg of a magnesium species such as elemental magnesium, or magnesium diglycinate or magnesium threonate, or a most preferred dosage range of from about 50 to about 180 mg (about 128 mg tested in Study 1 and about 90 mg in Study 2), including, but not limited to, from about 60 mg to about 290 mg, from about 70 mg to about 290 mg, from about 80 mg to about 290 mg, from about 90 mg to about 290 mg, from about 100 mg to about 290 mg, from about 100 mg to about 290 mg, from about 110 mg to about 290 mg, from about 120 mg to about 290 mg, from about 130 mg to about 290 mg, from about 50 to about 190 mg, from about 50 to about 200 mg, from about 50 to about 210 mg, from about 50 to about 220 mg, from about 50 to about 230 mg, from about 50 to about 240 mg, from about 50 to about 250 mg, from about 50 to about 260 mg, from about 50 to about 270 mg, from about 50 to about 280 mg, or from about 50 to about 290 mg of elemental magnesium.

The tested formulations (parenthetical above) were used in the intervention studies in the subsequent examples.

In an example, a method is contemplated to treat cramps, bloating, and/or dysmenorrhea including compositions that affect 4 or more of the following physiological pathways for the purpose of alleviating or diminishing pain associated with hormonal changes during the menstrual cycle, specifically surrounding menstruation, said composition including 5 or more active components selected from:

Plant extracts or nutritional supplements that stabilize mast cells or that are configured to stabilize mast cells, including, but not limited to, Vitamin D3, quercetin (and related flavonols such as but not limited to kaempferol), silybin (or silymarin) and/or resveratrol;

Plant extracts or nutritional supplements that inhibit the Cox 1/Cox2 pathway and/or reduce prostaglandin synthesis or that are configured to inhibit the Cox 1/Cox2 pathway and/or reduce prostaglandin synthesis, including, but not limited to, curcumin, resveratrol, boswellia, quercetin (and related flavonols), and/or gingerols;

Plant extracts or nutritional supplements that alleviate nutritional deficiencies or that are configured to alleviate nutritional deficiencies that in the general population are commonly linked to increased menstrual discomfort, including, but not limited to, Vitamin D, zinc, and/or magnesium;

Plant extracts or nutritional supplements that inhibit angiogenesis (through inhibition or reduction in the levels of matrix metalloproteinases) and/or prevent the growth of new blood vessels or that are configured to inhibit angiogenesis and/or prevent the growth of new blood vessels, including, but not limited to, green tea extract, resveratrol, quercetin, and/or silymarin;

Plant extracts or nutritional supplements that reduce oxidative stress through direct scavenging of free radicals, reduced iNOS, or increased glutathione, or that are configured to reduce oxidative stress, including, but not limited to, green tea catechins, resveratrol, quercetin, and/or N-acetyl cysteine;

Plant extracts or nutritional supplements that inhibit 5-lipoxygenase, or that are configured to inhibit 5-lipoxygenase, including, but not limited to, boswellia extract, and/or boswellic acids;

Plant extracts or mineral supplements that assist in smooth muscle relaxation or that are configured to assist in smooth muscle relaxation, including, but not limited to, magnesium;

Plant extracts that act as an antagonist of cannabinoid agonists and/or bind directly to cannabinoid receptors or that are configured to act as an antagonist of cannabinoid agonists and/or bind directly to cannabinoid receptors, including, but not limited to, phytocannabinoids (including cannabadiol, cannabichromene, cannabigerol), and/or diindolylmethane;

Soluble or insoluble dietary fiber that acts to absorb estrogen and assists with removal of estrogen from the body after estrogen is secreted into the intestines through the bile ducts, or that is configured to act to absorb estrogen and/or assist with removal of estrogen from the body, including, but not limited to, psyllium, and/or methylcellulose.

In certain examples, the dietary supplement further includes a component to improve bioavailability or configured to improve bioavailability. In certain examples, the component to improve bioavailability or configured to improve bioavailability is selected from the group consisting of a food complex, whole food matrix, delayed or controlled release technology; multi-compartment capsules or capsule-in-capsule technology, phytosome or liposome technology, cyclodextrin or other encapsulation technologies.

In certain examples, the components of the dietary supplement have been modified to complex with phospholipids of either vegetable or synthetic origin. In specific examples, these complexes are prepared by adding the phospholipid to the natural compound extract which has been dissolved in a protic solvent under reflux and with stirring.

The terms “complexed” and “encapsulated with phospholipids,” as used herein, unless stated otherwise, alone or in combination with other terms, refer to standard complexation and encapsulation procedures understood by a person of ordinary skill. In a specific example of “complexing,” or “encapsulation with phospholipids,” 20 g extract from turmeric rhizomes, having a curcuminoid content of >90% w/w, were dissolved in ethanol (800 ml) and the solution was refluxed. Sunflower phospholipids (80 g) were slowly added in portions, under reflux and with stirring. The resulting suspension was refluxed with stirring for 1 hr, then, while hot, concentrated under reduced pressure and finally dried in oven to leave a final product of 98 g composed of curcuminoids complexed with phospholipids. This process can be repeated with varying ratios of purified or enriched plant extracts with a ratio ranging from 10:1 phospholipid to extract, down to 3:2 phospholipid to extract.

The terms “standardized” and “standardization,” as used herein, unless stated otherwise, alone or in combination with other terms, refer to processes known to those of ordinary skill in the art applied to components of the dietary supplement to ensure that seasonal variation, or batch effects, or raw material sourcing, or other variations in the supply chain do not affect composition of the extracted materials, and that the ingredients maintain a specific ratio of active polyphenolic components.

In an example of standardization, the extract from the Boswellia serrata rhizome is measured using HPLC to ensure that triterpenoid acids compose more than 20% of the extract. To ensure consistency, extracts from multiple batches can be blended or combined following measurements to standardize the level and ratio of triterpenoid acids.

In an example of standardization for green tea catechins, catechins are extracted from the leaves of Camellia sinensis using a water-alcohol mixture. To compensate for seasonal or batch fluctuations in polyphenol levels, the extracts from multiple extract batches can be blended or combined following measurements to standardize the level and ratio of catechins. The extract is standardized to ensure >60% of the extract by mass was catechin polyphenols, and >60% of the catechin content was composed of EGCG.

In an example of standardization for curcuminoids, curcuminoids are extracted from the rhizome of Curcuma longa and purified using a water-ethanol mixture and concentrated under pressure. To compensate for batch fluctuations or variations in raw materials, the extracts from multiple extract batches can be blended or combined following measurements to standardize the level and ratio of curcuminoids. The extract is standardized to ensure >90% of the extract by mass is composed of curcuminoids.

In an example of standardization for quercetin, quercetin is extracted from the flower of Sophora japonica. To compensate for batch fluctuations or variations in raw materials, the extracts from multiple extract batches can be blended or combined following measurements to ensure >90% quercetin by weight.

In an example of standardization for withanolides, withanolides are extracted from Withania somnifera roots and leaves using an aqueous-alcohol mixture (for example water-ethanol or water-methanol). This extract is then dried and concentrated. To compensate for batch fluctuations or variations in raw materials, the extracts from multiple extract batches can be blended or combined following measurements to standardize and ensure >7% withanolide glycoside conjugates by weight, and >20% oligosaccharides by weight.

In an example of standardization for silybin, silybin is extracted from Silybum marianum fruit using an aqueous-alcohol mixture (for example water-ethanol or water-methanol). This extract is then dried and concentrated. To compensate for batch fluctuations or variations in growth conditions, the extracts from multiple extract batches can be blended or combined following measurements to standardize and ensure >70% silybin by weight.

In certain examples, the elemental magnesium has been modified to produce a chelate with an amino acid such as glycine, to produce magnesium diglycinate.

In certain examples, a magnesium-counter ion compound, such as magnesium threonate, is used.

In certain examples, the dietary supplement has been modified or configured to increase bioavailability through a reduction in particle size through micronization.

In certain examples, the dietary supplement further includes a component to extend the release kinetics. In certain examples, the component to extend release kinetics is selected from the group consisting of a food complex, whole food matrix, delayed or controlled release technology. In certain examples, cellulose, or similar bioadhesive polymers, may be used to protect the dietary supplement from the stomach pH and extend release kinetics. In certain examples, waxy substances, including, but not limited to Carnauba wax may be used to encapsulate the dietary supplement to extend release kinetics.

In certain examples, the dietary supplement is formulated as a once a day composition, a twice a day composition, a three times a day composition, or an every other day composition.

In certain examples, the dietary supplement is formulated to deliver the daily dose in a single dose. In certain examples, the dietary supplement is formulated to deliver a fraction of the daily dose and will be administered two or more times per day. In certain examples, the dietary supplement is formulated to deliver a dose equal to about half of a daily dose and will be administered twice per day. In certain examples, the dietary supplement is formulated to deliver a dose equal to about a third of a daily dose and will be administered three times per day. In some examples, the dose is administered every about 12 hours. In some examples, the dose is administered with every major meal.

In certain examples, the dietary supplement is formulated as a dosage form selected from the group consisting of a pill, a tablet, a caplet, a soft gelatin or vegetarian capsule, a hard gelatin or vegetarian capsule, a lozenge, a sachet, a cachet, a vegicap, a liquid drop, a liquid, a gel, a slurry, an elixir, a suspension, an emulsion, a solution, a dragee, and a syrup.

In certain examples, the dietary supplement is formulated as a chewable dosage form in which the dietary supplement has been included in a flavored or unflavored gummy that is easy to chew and swallow.

In certain examples, the dietary supplement is formulated as a dosage form intended for use as a rectal or vaginal suppository. The suppository form of the present invention can be prepared by adding the dietary supplement and other optional components, for example, a pH modifier, to a base component and molding the mixture into solid suppositories. Alternatively, the dietary supplement can be made into the form of suppositories encapsulated by soft gelatin or of injection type ointments.

In another example, a method of treating dysmenorrhea, and bloating, anxiety, depression, headache, backache, diarrhea, and/or constipation association with menstruation in a human subject includes the step of: administering to the human a composition including from about 30 milligrams to about 400 milligrams of Boswellia extract; from about 30 milligrams to about 400 milligrams of green tea catechins; from about 40 milligrams to about 500 milligrams of quercetin; and from about 50 milligrams to about 300 milligrams of magnesium. The composition may comprise Boswellia extract, green tea catechins, quercetin, and magnesium. Alternatively, the composition may consist essentially of Boswellia extract, green tea catechins, quercetin, and magnesium. Administration daily for at least 5 days, during which the human subject begins menstruating during a course of administration, may synergistically reduce or be configured to synergistically reduce symptoms of dysmenorrhea, as well as pelvic pain, bloating, anxiety, depression, headaches, backaches, constipation, and/or diarrhea associated with menstruation. The composition provides synergistic reduction of symptoms by: (1) inhibiting the cyclooxygenase pathway and reducing prostaglandin levels; (2) stabilizing mast cells and reducing histamine levels; (3) reducing the activity of 5-lipoxygenase and thus the level of lipoxygenase; (4) directly performing anti-oxidant scavenging and also upregulating the systemic antioxidant defenses; and (5) reducing a common nutritional deficiency associated with dysmenorrhea, and providing an ion linked to smooth muscle relaxation. In certain examples, the synergism exhibited by the combination of Boswellia extract, green tea catechins, quercetin, and magnesium may be such that doses of the individual ingredients would be sub-therapeutic if administered separately.

The composition may provide further augmented synergistic reduction in symptoms by further including one or more components selected from: from about 20 milligrams to about 300 milligrams of silybin; from about 30 milligrams to about 400 milligrams of resveratrol; from about 50 milligrams to about 600 milligrams of curcumin; from about 70 milligrams to about 600 milligrams of ashwagandha; and/or from about 1,000 to about 10,000 IU of Vitamin D. The composition may further comprise one or more components selected from silybin, resveratrol, curcumin, ashwagandha, and/or vitamin D. Alternatively, the composition may further consist essentially of one or more compounds selected from silybin, resveratrol, curcumin, ashwagandha, and/or Vitamin D.

In an example of a menstrual pain treatment system, a treatment system includes a single dosage form mixture of four or more components comprising: from about 30 milligrams to about 400 milligrams of Boswellia extract; from about 30 milligrams to about 400 milligrams of green tea catechins; from about 40 milligrams to about 500 milligrams of quercetin; and from about 50 milligrams to about 300 milligrams of magnesium. In another example, a mixture further comprises one or more components selected from: from about 20 milligrams to about 300 milligrams of silybin; from about 30 milligrams to about 400 milligrams of resveratrol; from about 50 milligrams to about 600 milligrams of curcumin; from about 70 milligrams to about 600 milligrams of ashwagandha; and/or from about 1,000 to about 10,000 IU Vitamin D.

Alternatively, in yet another example of a menstrual pain treatment system, a treatment system includes a single dosage form mixture of four or more components consisting essentially of: from about 30 milligrams to about 400 milligrams of Boswellia extract; from about 30 milligrams to about 400 milligrams of green tea catechins; from about 40 milligrams to about 500 milligrams of quercetin; and from about 50 milligrams to about 300 milligrams of magnesium. In another example, a mixture further consists essentially of one or more components selected from: from about 20 milligrams to about 300 milligrams of silybin; from about 30 milligrams to about 400 milligrams of resveratrol; from about 50 milligrams to about 600 milligrams of curcumin; from about 70 milligrams to about 600 milligrams of ashwagandha; and/or from about 1,000 to about 10,000 IU Vitamin D.

The methods described above may be further understood in connection with the following Examples. No limitation of the scope of the disclosure is intended by the illustration and description of certain examples of the disclosure. In addition, any alterations and/or modifications of the illustrated and/or described example(s) are contemplated as being within the scope of the present disclosure. Further, any other applications of the principles of the disclosure, as illustrated and/or described herein, as would normally occur to one skilled in the art to which the disclosure pertains, are contemplated as being within the scope of the present disclosure.

### Example 1

Menstrual Pain Study #1

Study type: Intervention

Enrollment: 12 participants

Allocation: All participants have both a baseline menstrual cycle, and an intervention menstrual cycle.

Intervention model: Crossover

Masking: Open-label

Intervention model description: This study was designed to test the effect of a dietary supplement on period pain when administered prior to and during menstruation. This study was an open-label crossover trial testing the effects of the dietary supplement formulation on dysmenorrhea.

Subjects: Females, ages 20-44 with self-reported dysmenorrhea.

Inclusion criteria: Healthy female of 20-44 years of age; regular menstrual cycles occurring every 21-35 days; self-reported dysmenorrhea (primary or secondary); willing to minimize nSAID usage during menstruation for the duration of the study.

Exclusion criteria: pregnant, or attempting to become pregnant.

Primary endpoints: (1) decreased pelvic pain; and (2) increased ability to engage in normal social/work engagements during menstruation.

Secondary endpoints: (1) reduced bloating; (2) improved mood; (3) reduced headaches; and (4) reduced intestinal issues.

Study methodology: Upon enrolling in the study, participants were sent a personal link to the online survey and requested to complete the survey within several days following the end of their next menstrual cycles. After completion of the survey, participants did not have access to the responses to reduce bias between months. Upon completion of the survey, participants were then sent a 7-day course of dietary supplements. In the subsequent menstrual cycle, participants were instructed to begin taking the supplements when: (1) the participants switched to placebo pills (or removed the NuvaRing) if the participants were using hormonal contraception; or (2) the participants began their periods. Participants were requested to take the pills with food at the same time every day. Participants completed an online survey within several days of the last day of their menstrual cycles.

Reporting: Using a standardized survey form, participants were asked to rank pain/discomfort levels according to an integer scale from 1 to 6, where 1 represents no pain, and 6 represents severe pain. Participants were requested to score: (1) Pain in the lower back during menstruation; (2) Pelvic pain (cramps, aches, or sharp pain) prior to menstruation—this was used as a control question to confirm a minimal bias between surveys; (3) Pelvic pain (cramps, aches, or sharp pains) during menstruation; (4) Intestinal issues (constipation, diarrhea, etc.) during menstruation; (5) Headaches or migraines during menstruation; (6) Bloating during menstruation; (7) Strength of negative emotions or moods during menstruation; (8) Number of cancelled activities (friends, exercise, outings, etc.) during the menstrual cycle (absolute number, not simple score from 1 to 6).

Statistical test: One-tailed, paired t-test using pre- and post-intervention results.

Dietary Supplement: The individual components of the dietary supplement as listed in Table 1 were obtained from suppliers Indena, Maypro, and Ixoreal. To increase bioavailability while minimizing the elemental dosage, all raw extracts (excluding Cholecalciferol and Ashwagandha) were either complexed with sunflower lecithin or micronized.

TABLE 1

| Supplement (Trade Name) - Supplier | Daily Dose (mg) | Elemental Dose (mg) |
|---|---|---|
| Sucrosomial Magnesium - Maypro | 400 | 128 |
| Resveratrol (MicroActive | 250 | 75 |
| Resveratrol) - Maypro | | |
| Silybin Phytosome | 150 | 60 |
| (Siliphos) - Indena | | |
| Green tea catechins (Green Select | 250 | 82.5 |
| Phytosome) - Indena | | |
| Curcumin Phytosome | 650 | 130 |
| (Meriva) - Indena | | |
| Quercetin Phytosome | 400 | 148 |
| (Quercefit) - Indena | | |
| Vitamin D3 | 0.1 | 4000 IU |
| Boswellia Phytosome | 250 | 82.5 |
| (Casperome) - Indena | | |
| Ashwagandha | 250 | 250 |
| (5% withanolides) - Ixoreal | | |

Results: Twelve participants enrolled, but two participants dropped out from the study following the completion of the first survey. One participant who dropped out was scheduled for fibroid surgery, and the other participant who dropped out decided to start in vitro fertilization (“IVF”) treatment. Data from these participants was excluded from the analysis. One participant found the pills hard to swallow, so the capsules were opened and added to food.

TABLE 2
Pain Scores Prior to Intervention (1 = no pain, 6 = severe pain)

| | | Pelvic Pain | Pelvic Pain | | | | | # |
|---|---|---|---|---|---|---|---|---|
| | | Before | During | Intestinal | | | Negative | Activities |
| Participant | Backaches | Period | Period | Issues | Headache | Bloating | Emotions | Cancelled |
| 1 | 2 | 2 | 3 | 3 | 3 | 4 | 2 | 0 |
| 2 | 3 | 2 | 3 | 1 | 4 | 4 | 3 | 1 |
| 3 | 1 | 1 | 5 | 3 | 1 | 6 | 4 | 2 |
| 4 | 1 | 5 | 5 | 4 | 5 | 4 | 3 | 3 |
| 5 | 1 | 2 | 2 | 1 | 1 | 4 | 4 | 0 |
| 6 | 2 | 3 | 5 | 3 | 1 | 4 | 3 | 1 |
| 7 | 3 | 4 | 5 | 5 | 3 | 6 | 6 | 1 |
| 8 | 1 | 1 | 2 | 4 | 5 | 5 | 5 | 3 |
| 9 | 5 | 3 | 5 | 3 | 5 | 6 | 5 | 2 |
| 10 | 3 | 2 | 3 | 3 | 1 | 3 | 6 | 4 |

TABLE 3
Pain Scores Post-Intervention (1 = no pain, 6 = severe pain)

| | | Pelvic Pain | Pelvic Pain | | | | | # |
|---|---|---|---|---|---|---|---|---|
| | | Before | During | Intestinal | | | Negative | Activities |
| Participant | Backaches | Period | Period | Issues | Headache | Bloating | Emotions | Cancelled |
| 1 | 1 | 1 | 2 | 2 | 1 | 2 | 2 | 0 |
| 2 | 2 | 1 | 2 | 1 | 4 | 1 | 1 | 0 |
| 3 | 1 | 2 | 1 | 2 | 1 | 1 | 1 | 0 |
| 4 | 1 | 3 | 3 | 2 | 5 | 3 | 4 | 0 |
| 5 | 2 | 4 | 3 | 3 | 1 | 2 | 1 | 0 |
| 6 | 1 | 1 | 4 | 3 | 1 | 3 | 1 | 0 |
| 7 | 1 | 4 | 1 | 2 | 1 | 3 | 6 | 0 |
| 8 | 3 | 3 | 3 | 4 | 1 | 5 | 5 | 0 |
| 9 | 5 | 3 | 5 | 6 | 4 | 4 | 5 | 2 |
| 10 | 2 | 2 | 2 | 1 | 1 | 1 | 3 | 2 |

Outcome—Primary endpoints: The study met the primary endpoints for both the number of activities cancelled by participants, and a reduction in period pain. (1) Prior to the intervention, participants reported a total of 17 activities that were cancelled during their first menstrual cycles. Following intervention, the incidence of cancelled activities was reduced to a total of 4 cancelled activities, and using a paired t-test, the reduction was statistically significant at the p<0.01 level. (2) Prior to the intervention, participants reported a total pelvic pain during menstruation of 28. The pelvic pain was reduced to a total of 16 during the intervention, which was a statistically significant reduction at the p<0.05 level.

Outcome—Secondary endpoints: The study met several, but not all of the secondary endpoints. (1) The intervention did statistically reduce headache or migraine pain levels. The total pain experienced from headaches or migraines was 19 pre-intervention, and 10 post-intervention. Using a paired t-test, this reduction was statistically significant at the p<0.05 level. (2) The intervention did statistically reduce bloating pain levels. The total pain experienced from bloating was 36 pre-intervention, and 15 post-intervention. Using a paired t-test, the reduction was statistically significant at the p<0.01 level. (3) The intervention did statistically reduce negative emotions. The total discomfort experienced from negative emotions (feeling overwhelmed, depressed, or anxious) was 31 pre-intervention, and 19 post-intervention. Using a paired t-test, the reduction was statistically significant at the p<0.05 level. (4) The intervention did not significantly reduce intestinal issues (for example, constipation, or diarrhea). Although the total pain experienced by participants went from 20 pre-intervention to 16 post-intervention, this was not statistically significant.

As a control, participants were asked about the level of pelvic pain experienced in the several days prior to the onset of menstruation. The responses were used to determine whether there was any significant bias between responses. The reduction in level of reported pelvic pain was not statistically significant between pre- and post-intervention. To calculate the total pain experienced by all subjects, the reported scores were adjusted to range from 0 (corresponding to no pain) to 5 (corresponding to extreme pain). The reported scores were then summed over all participants and shown below. For the pelvic pain prior to menstruation, the total pain levels changed from 15 pre-intervention to 14 post-intervention. The reduction indicates that the self-reported pain scores were consistent between surveys.

TABLE 4

| | | | | Intestine | | | Negative | Activities |
|---|---|---|---|---|---|---|---|---|
| | Pelvic Pain | Backache | Pelvic Pain | Issues | Headaches | Bloating | Emotions | Cancelled |
| | Before | During | During | During | During | During | During | During |
| | Period | Period | Period | Period | Period | Period | Period | Period |
| Total Pre- | 15 | 12 | 28 | 20 | 19 | 36 | 31 | 17 |
| Intervention | | | | | | | | |
| Pain | | | | | | | | |
| Total Post- | 14 | 9 | 16 | 16 | 10 | 15 | 19 | 4 |
| Intervention | | | | | | | | |
| Pain | | | | | | | | |
| P-value | 0.42 | 0.22 | 0.03 | 0.25 | 0.03 | 0.00 | 0.02 | 0.00 |

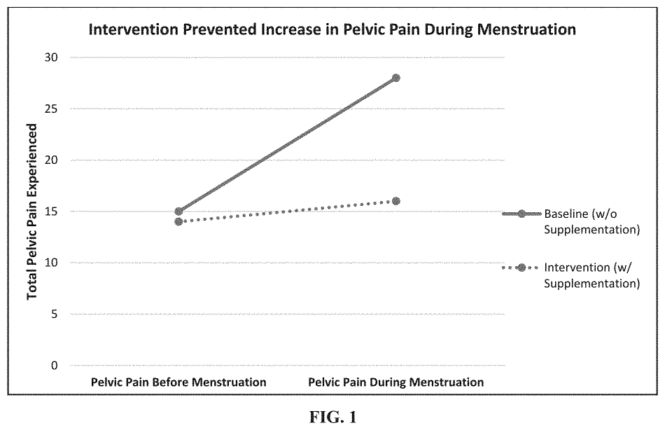

As expected, given that the participants had self-reported pain during menstruation, without intervention the total pelvic pain nearly doubled when participants began menstruation (from 15 prior to menstruation, to 28 during menstruation). This is a statistically significant increase (p=0.003). During the intervention, however, total pelvic pain only increased from 14 prior to menstruation to 16 during menstruation, which is not a statistically significant increase (p=0.36). The change in pain levels as a result of menstruation is demonstrated in the plot illustrated in FIG. 1.

### Example 2

Menstrual Pain Study #2

Study type: Intervention

Enrollment: 14 participants

Allocation: All participants have both a baseline menstrual cycle, and an intervention menstrual cycle.

Intervention model: Crossover

Masking: Open-label

Intervention model description: This study was designed to test the effect of a dietary supplement on period pain when administered prior to and during menstruation. This study was an open-label crossover trial testing the effects of the dietary supplement formulation on dysmenorrhea.

Subjects: Females, ages 20-44 with self-reported dysmenorrhea.

Inclusion criteria: Healthy female of 20-44 years of age; regular menstrual cycles occurring every 21-35 days; self-reported dysmenorrhea (primary or secondary); willing to minimize nSAID usage during menstruation for the duration of the study.

Exclusion criteria: pregnant, or attempting to become pregnant.

Primary endpoints: (1) decreased pelvic pain; and (2) increased ability to engage in normal social/work engagements during menstruation.

Secondary endpoints: (1) reduced bloating; (2) improved mood; (3) reduced headaches; and (4) reduced intestinal issues.

Study methodology: Upon enrolling in the study, participants were sent a personal link to the online survey and requested to complete the survey within several days following the end of their next menstrual cycles. After completion of the survey, participants did not have access to the responses to reduce bias between months. Upon completion of the survey, participants were then sent a 7-day course of dietary supplements that were to be taken twice a day. In the subsequent menstrual cycle, participants were instructed to begin taking the supplements when: (1) the participants switched to placebo pills (or removed the NuvaRing) if the participants were using hormonal contraception; or (2) the participants began their periods. Participants were requested to take the pills with food at the same time every day (once in the morning and once in the evening). Participants completed an online survey within several days of the last day of their menstrual cycles.

Reporting: Using a standardized survey form, participants were asked to rank pain/discomfort levels according to an integer scale from 1 to 6, where 1 represents no pain, and 6 represents severe pain. Participants were requested to score: (1) Pelvic pain (cramps, aches, or sharp pain) during menstruation; (2) Intestinal issues (constipation, diarrhea, etc.) during menstruation; (3) Headaches or migraines during menstruation; (4) Bloating during menstruation; (5) Strength of negative emotions or moods during menstruation; (6) Number of cancelled activities (friends, exercise, outings, etc.) during the menstrual cycle (absolute number, not simple score from 1 to 6).

Statistical test: Two-tailed, paired t-test using pre- and post-intervention results. Significance was determined using the p<0.01 threshold.

Total Pain Calculation: To adjust for the survey results where no pain was ranked at a value of 1, when calculating the total discomfort, each response was normalized by subtracting 1, and then summed. For example, if all participants reported that intestinal discomfort was at a value of 1 (no pain), then the total intestinal pain for all participants would be 0.

Dietary Supplement: the individual components of the dietary supplement were obtained from Indena, Maypro, and Natreon. To increase bioavailability while minimizing the elemental dosage, all raw extracts (excluding Cholecalciferol, Ashwagandha, and Resveratrol) were complexed with sunflower lecithin.

TABLE 5

| Supplement (Trade Name) - Supplier | Daily Dose (mg) | Elemental Dose (mg) |
|---|---|---|
| Sucrosomial Magnesium - Maypro | 400 | 90 |
| Polygonum cuspidatum root extract | 252 | 247 |
| (Standardized for 98% trans-Resveratrol) | | |
| Silybin Phytosome (Siliphos: Providing | 150 | 44.5 |
| NLT 29.7% of Silybin) - Indena | | |
| Green tea catechins (Green Select | 252 | 120 |
| Phytosome: Standardized for 19-25% | | |
| Catechins and 25% EGCG) - Indena | | |
| Curcumin Phytosome (Meriva: Standardized | 648 | 117 |
| for 18-22% Curcuminoids) - Indena | | |
| Quercetin Phytosome (Quercefit: | 282 | 96 |
| Standardized for 34-42% Quercetin) - | | |
| Indena | | |
| Vitamin D3 | N/A | 4800 IU |
| Boswellia Phytosome (Casperome: | 252 | 63 |
| Standardized for NLT 25% total boswellic | | |
| acids and lupeolics acids) - Indena | | |
| Ashwagandha (Sensoril: Standardized for | 132 | 13.2 |
| NLT 10% withanolide glycoside | | |
| conjugates) - Natreon | | |

Results: All fourteen participants that enrolled completed the trial.

TABLE 6
Pain Scores Prior to Intervention (1 = no pain, 6 = severe pain)

| | Pelvic Pain | | | | | # |
|---|---|---|---|---|---|---|
| | During | Intestinal | | | Negative | Activities |
| Participant | Period | Issues | Headache | Bloating | Emotions | Cancelled |
| 1 | 6 | 4 | 4 | 6 | 6 | 5 |
| 2 | 3 | 3 | 1 | 4 | 3 | 2 |
| 3 | 6 | 6 | 3 | 5 | 5 | 4 |
| 4 | 1 | 1 | 1 | 5 | 4 | 0 |
| 5 | 5 | 6 | 2 | 3 | 3 | 0 |
| 6 | 6 | 6 | 3 | 6 | 5 | 2 |
| 7 | 4 | 4 | 2 | 5 | 5 | 1 |
| 8 | 5 | 4 | 1 | 4 | 4 | 5 |
| 9 | 4 | 5 | 1 | 6 | 5 | 2 |
| 10 | 5 | 6 | 5 | 5 | 4 | 0 |
| 11 | 4 | 5 | 3 | 5 | 4 | 3 |
| 12 | 6 | 2 | 2 | 4 | 5 | 2 |
| 13 | 6 | 5 | 1 | 3 | 2 | 2 |
| 14 | 5 | 6 | 4 | 6 | 4 | 1 |

TABLE 7
Pain Scores Post-Intervention (1 = no pain, 6 = severe pain)

| | Pelvic Pain | | | | | # |
|---|---|---|---|---|---|---|
| | During | Intestinal | | | Negative | Activities |
| Participant | Period | Issues | Headache | Bloating | Emotions | Cancelled |
| 1 | 3 | 2 | 1 | 5 | 2 | 0 |
| 2 | 1 | 1 | 1 | 2 | 1 | 0 |
| 3 | 4 | 1 | 1 | 4 | 3 | 0 |
| 4 | 1 | 2 | 1 | 2 | 1 | 0 |
| 5 | 3 | 2 | 1 | 3 | 3 | 0 |
| 6 | 2 | 1 | 1 | 2 | 1 | 0 |
| 7 | 2 | 2 | 1 | 3 | 3 | 0 |
| 8 | 2 | 1 | 1 | 1 | 1 | 0 |
| 9 | 2 | 3 | 1 | 4 | 4 | 2 |
| 10 | 2 | 3 | 2 | 3 | 3 | 0 |
| 11 | 1 | 2 | 2 | 2 | 3 | 1 |
| 12 | 3 | 1 | 1 | 2 | 3 | 1 |
| 13 | 3 | 4 | 1 | 2 | 2 | 1 |
| 14 | 3 | 4 | 2 | 3 | 2 | 0 |

Outcome—Primary endpoints: The study met the primary endpoints for both the number of activities cancelled by participants, and a reduction in pelvic pain. (1) Prior to the intervention, participants reported that a total of 29 activities were cancelled during their first menstrual cycles. Following intervention, the incidence of cancelled activities was reduced to a total of 5 cancelled activities, and using a paired t-test, the reduction was statistically significant at the p<0.01 level. (2) Prior to the intervention, participants reported a total pelvic pain during menstruation of 52. The pelvic pain was reduced to a total of 18 during the intervention, which was a statistically significant reduction at the p<0.01 level.

Outcome—Secondary endpoints: The study met all of the secondary endpoints. (1) The intervention did statistically reduce headache or migraine pain levels. The total pain experienced from headaches or migraines was 19 pre-intervention, and 3 post-intervention. Using a paired t-test, this reduction was statistically significant at the p<0.01 level. (2) The intervention did statistically reduce bloating pain levels. The total pain experienced from bloating was 53 pre-intervention, and 24 post-intervention. Using a paired t-test, the reduction was statistically significant at the p<0.01 level. (3) The intervention did statistically reduce negative emotions. The total discomfort experienced from negative emotions (feeling overwhelmed, depressed, or anxious) was 45 pre-intervention, and 18 post-intervention. Using a paired t-test, the reduction was statistically significant at the p<0.01 level. The intervention did statistically reduce intestinal issues (constipation, diarrhea, etc.). The total pain experienced by participants went from 49 pre-intervention to 15 post-intervention. Using a paired t-test, this was significant at the p<0.01 level.

To calculate the total pain experienced by all subjects, the reported scores were adjusted to range from 0 (corresponding to no pain) to 5 (corresponding to extreme pain). The reported scores were then summed over all participants and the results are shown below.

TABLE 8

| | | Intestinal | | | Negative | Activities |
|---|---|---|---|---|---|---|
| | Pelvic Pain | Issues | Headaches | Bloating | Emotions | Cancelled |
| | During | During | During | During | During | During |
| | Period | Period | Period | Period | Period | Period |
| Total Pre- | 52 | 49 | 19 | 53 | 45 | 29 |
| Intervention | | | | | | |
| Pain | | | | | | |
| Total Post- | 18 | 15 | 3 | 24 | 18 | 5 |
| Intervention | | | | | | |
| Pain | | | | | | |
| P-value | 0.00 | 0.00 | 0.00 | 0.00 | 0.00 | 0.00 |

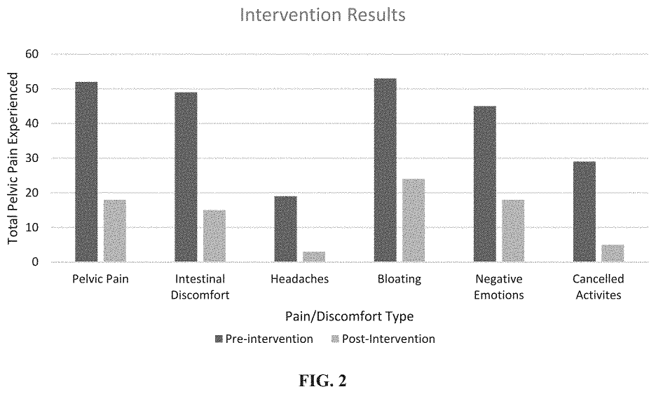

The intervention significantly improved all discomfort scores as can clearly be seen when plotted in bar graph form, as illustrated by FIG. 2. All plotted results are statistically significant at the P<0.01 threshold using a two-tailed t-test.

The use of the terms “a,” “an,” “the,” and similar referents in the context of describing the presently claimed invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. As stated hereinabove, use of the term “about” is intended to describe values either above or below the stated value in a range of approximately ±10%; in other examples, the values may range in value either above or below the stated value in a range of approximately ±5%; in other examples, the values may range in value either above or below the stated value in a range of approximately ±2%; in other examples, the values may range in value either above or below the stated value in a range of approximately ±1%. The preceding ranges are intended to be made clear by context, and no further limitation is implied. All methods described herein can be performed in any suitable order unless otherwise indicated herein or otherwise clearly contradicted by context. The use of any and all examples, or exemplary language (for example, “such as”) provided herein, is intended merely to better illuminate the invention and does not pose a limitation on the scope of the invention unless otherwise claimed. No language in the specification should be construed as indicating any non-claimed element as essential to the practice of the invention.

To clarify the use of and to hereby provide notice to the public, the phrases “at least one of <A>, <B>, . . . and <N>” or “at least one of <A>, <B>, . . . <N>, or combinations thereof” of “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group including A, B, . . . and N. In other words, the phrases mean any combination of one or more of the elements A, B, . . . or N including any one element alone or the one element in combination with one or more of the other elements which may also include, in combination, additional elements not listed. Unless otherwise indicated or the context suggests otherwise, as used herein, “a” or “an” means “at least one” or “one or more.”

While in the foregoing specification this invention has been described in relation to certain examples thereof, and many details have been put forth for the purpose of illustration, it will be apparent to those skilled in the art that the invention is susceptible to additional examples and that certain of the details described herein can be varied considerably without departing from the basic principles of the invention.

All references cited herein are incorporated by reference in their entireties. The present invention may be exemplified in other specific forms without departing from the spirit or essential attributes thereof and, accordingly, reference should be made to the appended claims, rather than to the foregoing specification, as indicating the scope of the invention.

The subject-matter of the disclosure may also relate, among others, to the following aspects:

A first aspect relates to a method of treating dysmenorrhea and/or pain associated therewith in a human, comprising: administering to the human a composition comprising four or more components comprising a total combined daily dose of: from about 30 milligrams to about 400 milligrams of Boswellia extract; from about 30 milligrams to about 400 milligrams of green tea catechins; from about 40 milligrams to about 500 milligrams of quercetin; and from about 50 milligrams to about 300 milligrams of magnesium; wherein the composition is administered for at least 5 days; wherein the human begins menstruating during a course of administration; and wherein the four or more components act synergistically to treat dysmennorhea.

A second aspect relates to the method of claim 1, wherein the composition further comprises one or more components selected from: from about 20 milligrams to about 300 milligrams of silybin; from about 30 milligrams to about 400 milligrams of resveratrol; from about 50 milligrams to about 600 milligrams of curcumin; from about 70 milligrams to about 600 milligrams of ashwagandha; and/or from about 1,000 to about 10,000 IU of Vitamin D.

A third aspect relates to a method of treating dysmenorrhea and/or pain associated therewith in a human, comprising: administering to the human a composition comprising a mixture of Boswellia extract, green tea catechins, quercetin, magnesium, silybin, resveratrol, curcumin, ashwagandha, and Vitamin D.

A fourth aspect relates to the method of any preceding aspect, wherein the composition is administered for at least 10 days during a course of administration.

A fifth aspect relates to the method of any preceding aspect, wherein the composition is administered for two or more consecutive menstrual cycles.

A sixth aspect relates to the method of any preceding aspect, wherein the composition further comprises one or more components selected from: a phytocannabinoid, diindolylmethane, psyllium, and methylcellulose.

A seventh aspect relates to the method of any preceding aspect, wherein the composition comprises: from about 40 milligrams to about 140 milligrams of Boswellia extract; from about 40 milligrams to about 200 milligrams of green tea catechins; from about 50 milligrams to about 200 milligrams of quercetin; and from about 50 milligrams to about 180 milligrams of magnesium.

An eighth aspect relates to the method of any preceding aspect, wherein the composition comprises: about 83 milligrams of Boswellia extract; about 83 milligrams of green tea catechins; about 148 milligrams of quercetin; and about 128 milligrams of magnesium.

A ninth aspect relates to the method of any of aspects 1 to 7, wherein the composition comprises: about 63 milligrams of Boswellia extract; about 120 milligrams of green tea catechins; about 96 milligrams of quercetin; and about 90 milligrams of magnesium.

A tenth aspect relates to the method of any preceding aspects, wherein the composition further comprises one or more components selected from: from about 20 milligrams to about 80 milligrams of silybin; from about 40 milligrams to about 300 milligrams of resveratrol; from about 70 milligrams to about 200 milligrams of curcumin; from about 80 milligrams to about 400 milligrams of ashwagandha; and/or from about 2,000 to about 7,000 IU of Vitamin D.

An eleventh aspect relates to a method of treating bloating, headache, backache, anxiety, depression, diarrhea, and/or constipation associated with menstruation in a human, comprising: administering to the human a composition comprising four or more components comprising a total combined daily dose of: from about 30 milligrams to about 400 milligrams of Boswellia extract; from about 30 milligrams to about 400 milligrams of green tea catechins; from about 40 milligrams to about 500 milligrams of quercetin; and from about 50 milligrams to about 300 milligrams of magnesium; wherein the composition is administered daily for at least 5 days; wherein the human begins menstruating during the course of administration; and wherein the four or more components act synergistically to treat bloating, headache, backache, anxiety, depression, diarrhea, and/or constipation.

A twelfth aspect relates to the method of aspect 11, wherein the composition further comprises one or more components selected from: from about 20 milligrams to about 300 milligrams of silybin; from about 30 milligrams to about 400 milligrams of resveratrol; from about 50 milligrams to about 600 milligrams of curcumin; from about 70 milligrams to about 600 milligrams of ashwagandha; and/or from about 1,000 to about 10,000 IU of Vitamin D.

A thirteenth aspect relates to the method of aspects 11 and 12, wherein the composition is administered for at least 10 days.

A fourteenth aspect relates to the method of any of aspects 11 to 13, wherein the composition is administered for two or more consecutive menstrual cycles.

A fifteenth aspect relates to the method of any of aspects 11 to 14, wherein the composition further comprises one or more components selected from: a phytocannabinoid, diindolylmethane, psyllium, and methycellulose.

A sixteenth aspect relates to the method of any of aspects 11 to 15, wherein the composition comprises: from about 40 milligrams to about 140 milligrams of Boswellia extract; from about 40 milligrams to about 200 milligrams of green tea catechins; from about 50 milligrams to about 200 milligrams of quercetin; and from about 50 milligrams to about 180 milligrams of magnesium.

A seventeenth aspect relates to the method of any of aspects 11 to 16, wherein the composition comprises: about 83 milligrams of Boswellia extract; about 83 milligrams of green tea catechins; about 148 milligrams of quercetin; and about 128 milligrams of magnesium.

An eighteenth aspect relates to the method of any of aspects 11 to 16, wherein the composition comprises: about 63 milligrams of Boswellia extract; about 120 milligrams of green tea catechins; about 96 milligrams of quercetin; and about 90 milligrams of magnesium.

A nineteenth aspect relates to the method of any of aspects 11 to 18, wherein the composition further comprises one or more components selected from: from about 20 milligrams to about 80 milligrams of silybin; from about 40 milligrams to about 300 milligrams of resveratrol; from about 70 milligrams to about 200 milligrams of curcumin; from about 80 milligrams to about 400 milligrams of ashwagandha; and/or from about 2,000 to about 7,000 IU of Vitamin D.

A nineteenth aspect relates to a menstrual pain treatment system, comprising: a single dosage form mixture of four or more components comprising: from about 30 milligrams to about 400 milligrams of Boswellia extract that has been standardized, and complexed or encapsulated with phospholipids; from about 30 milligrams to about 400 milligrams of green tea catechins that has been standardized, and complexed or encapsulated with phospholipids; from about 40 milligrams to about 500 milligrams of quercetin that has been standardized, and complexed or encapsulated with phospholipids; and from about 50 milligrams to about 300 milligrams of magnesium that has been standardized, and complexed or encapsulated with phospholipids, or from about 50 milligrams to about 300 milligrams of magnesium diglycinate or magnesium threonate; and wherein synergistic activity of the mixture is configured to cause, in a menstruating human: a reduction in pelvic pain associated with menstruation; a reduction in pelvic pain associated with menstruation; a reduction in bloating associated with menstruation; a reduction in symptoms of anxiety and/or depression associated with menstruation; a reduction in headaches associated with menstruation; a reduction in backaches associated with menstruation; and/or a reduction in constipation or diarrhea associated with menstruation.

A twentieth aspect relates to the menstrual pain treatment system of aspect 19, wherein the mixture further comprises one or more components selected from: from about 20 milligrams to about 300 milligrams of silybin that has been standardized, and complexed or encapsulated with phospholipids; from about 30 milligrams to about 400 milligrams of resveratrol; from about 50 milligrams to about 400 milligrams of curcumin that has been standardized, and complexed or encapsulated with phospholipids; from about 70 milligrams to about 600 milligrams of ashwagandha; and/or from about 1,000 to about 10,000 IU Vitamin D.

In addition to the features mentioned in each of the independent aspects enumerated above, some examples may show, alone or in combination, the optional features mentioned in the dependent aspects and/or as disclosed in the description above and shown in the figures.

What is claimed is:

1. A method of treating dysmenorrhea and/or pain associated therewith in a human, comprising:
administering to the human a composition comprising four or more components comprising a total combined daily dose of:
from about 30 milligrams to about 400 milligrams of Boswellia extract;
from about 30 milligrams to about 400 milligrams of green tea catechins;
from about 40 milligrams to about 500 milligrams of quercetin; and
from about 50 milligrams to about 300 milligrams of magnesium;
wherein the composition is administered for at least 5 days;
wherein the human begins menstruating during a course of administration; and
wherein the four or more components act synergistically to treat dysmenorrhea.

2. The method of claim 1, wherein the composition further comprises one or more components selected from:
from about 20 milligrams to about 300 milligrams of silybin;
from about 30 milligrams to about 400 milligrams of resveratrol;
from about 50 milligrams to about 600 milligrams of curcumin;
from about 70 milligrams to about 600 milligrams of ashwagandha; and/or
from about 1,000 to about 10,000 IU of Vitamin D.

3. The method of claim 1, wherein the composition is administered for at least 10 days during a course of administration.

4. The method of claim 1, wherein the composition is administered for two or more consecutive menstrual cycles.

5. The method of claim 1, wherein the composition further comprises one or more components selected from: a phytocannabinoid, diindolylmethane, psyllium, and methylcellulose.

6. The method of claim 1, wherein the composition comprises:
from about 40 milligrams to about 140 milligrams of Boswellia extract;
from about 40 milligrams to about 200 milligrams of green tea catechins;
from about 50 milligrams to about 200 milligrams of quercetin; and
from about 50 milligrams to about 180 milligrams of magnesium.

7. The method of claim 1, wherein the composition comprises:
about 83 milligrams of Boswellia extract;
about 83 milligrams of green tea catechins;
about 148 milligrams of quercetin; and
about 128 milligrams of magnesium.

8. The method of claim 1, wherein the composition comprises:
about 63 milligrams of Boswellia extract;
about 120 milligrams of green tea catechins;
about 96 milligrams of quercetin; and
about 90 milligrams of magnesium.

9. The method of claim 1, wherein the composition further comprises one or more components selected from:
from about 20 milligrams to about 80 milligrams of silybin;
from about 40 milligrams to about 300 milligrams of resveratrol;
from about 70 milligrams to about 200 milligrams of curcumin;
from about 80 milligrams to about 400 milligrams of ashwagandha; and/or
from about 2,000 to about 7,000 IU of Vitamin D.

10. A method of treating bloating, headache, backache, anxiety, depression, diarrhea, and/or constipation associated with menstruation in a human, comprising:
administering to the human a composition comprising four or more components comprising a total combined daily dose of:
from about 30 milligrams to about 400 milligrams of Boswellia extract;
from about 30 milligrams to about 400 milligrams of green tea catechins;
from about 40 milligrams to about 500 milligrams of quercetin; and
from about 50 milligrams to about 300 milligrams of magnesium;
wherein the composition is administered daily for at least 5 days;
wherein the human begins menstruating during the course of administration; and
wherein the four or more components act synergistically to treat bloating, headache, backache, anxiety, depression, diarrhea, and/or constipation.

11. The method of claim 10, wherein the composition further comprises one or more components selected from:
from about 20 milligrams to about 300 milligrams of silybin;
from about 30 milligrams to about 400 milligrams of resveratrol;
from about 50 milligrams to about 600 milligrams of curcumin;
from about 70 milligrams to about 600 milligrams of ashwagandha; and/or
from about 1,000 to about 10,000 IU of Vitamin D.

12. The method of claim 10, wherein the composition is administered for at least 10 days.

13. The method of claim 10, wherein the composition is administered for two or more consecutive menstrual cycles.

14. The method of claim 10, wherein the composition further comprises one or more components selected from: a phytocannabinoid, diindolylmethane, psyllium, and methylcellulose.

15. The method of claim 10, wherein the composition comprises:
from about 40 milligrams to about 140 milligrams of Boswellia extract;
from about 40 milligrams to about 200 milligrams of green tea catechins;
from about 50 milligrams to about 200 milligrams of quercetin; and
from about 50 milligrams to about 180 milligrams of magnesium.

16. The method of claim 10, wherein the composition comprises:
about 83 milligrams of Boswellia extract;
about 83 milligrams of green tea catechins;
about 148 milligrams of quercetin; and
about 128 milligrams of magnesium.

17. The method of claim 10, wherein the composition comprises:
about 63 milligrams of Boswellia extract;
about 120 milligrams of green tea catechins;
about 96 milligrams of quercetin; and
about 90 milligrams of magnesium.

18. The method of claim 10, wherein the composition further comprises one or more components selected from:
from about 20 milligrams to about 80 milligrams of silybin;
from about 40 milligrams to about 300 milligrams of resveratrol;
from about 70 milligrams to about 200 milligrams of curcumin;
from about 80 milligrams to about 400 milligrams of ashwagandha; and/or
from about 2,000 to about 7,000 IU of Vitamin D.

19. A menstrual pain treatment system, comprising:
a single dosage form mixture of four or more components comprising:
from about 30 milligrams to about 400 milligrams of Boswellia extract that has been standardized, and complexed or encapsulated with phospholipids;
from about 30 milligrams to about 400 milligrams of green tea catechins that has been standardized, and complexed or encapsulated with phospholipids;
from about 40 milligrams to about 500 milligrams of quercetin that has been standardized, and complexed or encapsulated with phospholipids; and
from about 50 milligrams to about 300 milligrams of magnesium that has been standardized, and complexed or encapsulated with phospholipids, or from about 50 milligrams to about 300 milligrams of magnesium diglycinate or magnesium threonate; and
wherein synergistic activity of the mixture is configured to cause, in a menstruating human:
a reduction in pelvic pain associated with menstruation;
a reduction in bloating associated with menstruation;
a reduction in symptoms of anxiety and/or depression associated with menstruation;
a reduction in headaches associated with menstruation;
a reduction in backaches associated with menstruation; and/or
a reduction in constipation or diarrhea associated with menstruation.

20. The treatment system of claim 19, wherein the mixture further comprises one or more components selected from:
from about 20 milligrams to about 300 milligrams of silybin that has been standardized, and complexed or encapsulated with phospholipids;
from about 30 milligrams to about 400 milligrams of resveratrol;
from about 50 milligrams to about 600 milligrams of curcumin that has been standardized, and complexed or encapsulated with phospholipids;
from about 70 milligrams to about 600 milligrams of ashwagandha; and/or
from about 1,000 to about 10,000 IU Vitamin D.

21. A method of treating dysmenorrhea and/or pain associated therewith in a human, comprising:
administering to the human a composition comprising a mixture of Boswellia extract, green tea catechins, quercetin, magnesium, silybin, resveratrol, curcumin, ashwagandha, and Vitamin D;
wherein the composition is administered for at least 5 days;
wherein the human begins menstruating during a course of administration; and
wherein the four or more components act synergistically to treat dysmenorrhea.

22. The method of claim 21, wherein the composition comprises a mixture of:
from about 30 milligrams to about 400 milligrams of Boswellia extract;
from about 30 milligrams to about 400 milligrams of green tea catechins;
from about 40 milligrams to about 500 milligrams of quercetin;
from about 50 milligrams to about 300 milligrams of magnesium;
from about 20 milligrams to about 300 milligrams of silybin;
from about 30 milligrams to about 400 milligrams of resveratrol;
from about 50 milligrams to about 600 milligrams of curcumin;
from about 70 milligrams to about 600 milligrams of ashwagandha; and
from about 1,000 to about 10,000 IU of Vitamin D.

23. The method of claim 21, wherein the composition is administered for at least 10 days during a course of administration.

24. The method of claim 21, wherein the composition is administered for two or more consecutive menstrual cycles.

25. The method of claim 21, wherein the composition further comprises one or more components selected from: a phytocannabinoid, diindolylmethane, psyllium, and methylcellulose.

26. The method of claim 21, wherein the composition comprises a mixture of:
from about 40 milligrams to about 140 milligrams of Boswellia extract;
from about 40 milligrams to about 200 milligrams of green tea catechins;
from about 50 milligrams to about 200 milligrams of quercetin;
from about 50 milligrams to about 180 milligrams of magnesium;
from about 20 milligrams to about 80 milligrams of silybin;
from about 40 milligrams to about 300 milligrams of resveratrol;
from about 70 milligrams to about 200 milligrams of curcumin;
from about 80 milligrams to about 400 milligrams of ashwagandha; and
from about 2,000 to about 7,000 IU of Vitamin D.
